(19) United States
(12) Patent Application Publication
(10) Pub. No.: US 2026/0224832 A1
(43) Pub. Date: Aug. 6, 2026

(54) Respiratory therapy apparatus delivering intermittent positive pressure breathing assistance and decreasing insufflation flowrate

(71) Applicant: EOVE, PAU (FR)

(72) Inventors: Cedric JOURDAIN, PAU (FR); Tingting LIU, PAU (FR); Marie-Amedee GALBRUN, PAU (FR); Manuel TEILLET, PAU (FR)

(21) Appl. No.: 19/443,530

(22) Filed: Jan. 8, 2026

(30) Foreign Application Priority Data
Jan. 23, 2025 (FR) 2500694

(51) Int. Cl. A61M 16/00; A61M 16/20

(52) U.S. Cl. CPC A61M 16/0069; A61M 16/0003; A61M 16/208; A61M 2016/0027; A61M 2016/0039; A61M 2205/3365; A61M 2205/42

(57) ABSTRACT

The invention relates to a respiratory therapy apparatus delivering intermittent positive pressure breathing assistance (IPPB) to an individual, comprising operating means (4) which are configured so that, during each inspiratory phase of the individual, they command the turbine of the apparatus to supply a regulated gas flowrate that decreases, over the course of a non-zero gas-insufflation duration (Di) that is sufficient to reach a fixed maximum-pressure threshold (Pₘₐₓ).

### CROSS REFERENCE TO RELATED APPLICATION

This patent application claims priority to French Application No. 2500694, filed on Jan. 23, 2025 and the entire contents of which are incorporated herein by reference.

### TECHNICAL FIELD

The invention relates to a respiratory therapy apparatus delivering Intermittent Positive Pressure Breathing (or IPPB) assistance, also known as an “IPPB apparatus”, which employs a regulated gas flowrate that decreases, over the course of a non-zero gas-insufflation duration that is sufficient to reach a fixed maximum-pressure threshold.

### BACKGROUND

In general, providing respiratory therapy of the IPPB type (hereinafter referred to as “IPPB therapy”) to certain individuals, also known as “patients”, who are suffering from respiratory problems or conditions, notably makes it possible to improve:
bronchial drainage by delivering a high inspiratory volume that makes it possible to increase the effectiveness of the expiratory flow and of coughing;
the respiratory function by exceeding the patient's maximum inspiration and thus increasing their vital capacity; and
lung recruitment, particularly recruitment of those regions of the lungs that are poorly ventilated or unventilated.

For example, EP4079356 teaches an apparatus to assist with coughing enabling gas insufflation to be given to patients suffering from respiratory problems who need help voiding their pulmonary secretions and/or to improve the elasticity of their lungs and thorax, whether these patients be adult or paediatric patients, and which apparatus can be used with ease by an individual having little or no medical knowledge, typically a carer, someone from the patient's family circle, also known as “the helpers”.

Other respiratory therapy apparatuses are described in DE102014001218, WO2015/110098, EP1502619, US2013/047983, US2024/165351 and US2008/000478.

In general, IPPB therapy relies on the application of a gas flowrate that is constant during inspiration until a given maximum pressure is reached, followed by expiration with or without positive expiratory pressure (PEP). Synchronization with the respiratory phases of the patient is achieved by detecting the effort made by the patient, typically the demand for gas at the start of inspiration, or by using a mechanism or device for manually triggering inspiration, for example by pressing a button or a command key.

There are many types of respiratory therapy device or apparatus that enable IPPB therapy to be given, but these all have disadvantages of varying levels of severity.

At the present time, IPPB therapy devices operate on the basis of regulating the inspiration flowrate, namely the flowrate of gas (e.g. air) supplied to the airways of the patient while they are being treated. The flowrate supplied by the gas source, such as a turbine or the like, of the gas-supplying device or apparatus, is usually constant during each inspiratory phase of the patient, leading to a flowrate curve that is rectangular in shape. Keeping the supplied flowrate constant allows the patient's lungs to fill gradually with a simultaneous gradual increase in the gas pressure in the airways, particularly in the lungs, of the patient.

The/each inspiratory phase of the patient ends when a previously-fixed maximum pressure, i.e. a settable maximum-pressure threshold, is reached. In other words, to end inspiration (i.e. an inspiratory phase), use is made of the pressure threshold that has been set by the user, typically someone from the medical-care team, and which, when reached, triggers a switch to expiration (i.e. an expiratory phase). This approach aims to achieve better control over the maximum cutoff pressure so as to maximize patient comfort and limit the risks of barotrauma.

In general, the objective of the treatment session is to obtain a maximum volume in the patient's lungs, and increasing the cutoff pressure is one way of achieving this.

However, it has been found in practice that the effectiveness of the treatment and/or the comfort of the patients as well as adherence to the treatment, are notably dependent on the physiology of the patients themselves, notably in terms of compliance and resistance, but also on the type of breathing interface used for administering the gas, i.e. breathing mask with or without leakage.

One problem is therefore to propose a respiratory therapy device or apparatus able to deliver intermittent positive pressure breathing assistance, namely therapy of the IPPB type, or an “IPPB apparatus”, that is improved, and notably that is able to overcome all or some of the aforementioned disadvantages and in particular to deliver IPPB therapy that is more effective with improved patient comfort while at the same time improving the filling of the patient's lungs with the insufflated gas, or even also the patient's adherence to the treatment.

### SUMMARY

One solution according to the invention is a respiratory therapy apparatus (i.e. device or installation), namely an “IPPB apparatus”, for supplying intermittent positive pressure breathing (IPPB) assistance to an individual, i.e. a patient, and comprising:
a peripheral casing or shell,
a motorized turbine (i.e. a turbine, compressor, (micro) blower or the like) to supply a respiratory gas under pressure,
an internal gas circuit for conveying the respiratory gas delivered by the turbine,
a flowrate sensor arranged on the internal gas circuit,
a nonreturn device arranged in the internal gas circuit downstream of the flowrate sensor,
an external patient circuit that can be fluidically connected to the internal gas circuit to convey the respiratory gas coming from the internal gas circuit,
a proximal pressure sensor arranged to enable measurement of the pressure (i.e. to take instantaneous-pressure measurements) within the external patient circuit near to the expiratory valve, and
operating means connected to the proximal pressure sensor and to the motorized turbine and configured to command the turbine as a function of at least one pressure measurement taken by the proximal pressure sensor and of at least one flowrate measurement taken by the flowrate sensor.

The turbine, the operating means, the internal circuit, the flowrate sensor and the nonreturn device are arranged in the peripheral casing or shell.

In addition, in the IPPB apparatus, the operating means are configured so that, during each inspiratory phase of the individual, i.e. of the patient, they command the turbine to supply a regulated gas flowrate that decreases, over the course of a non-zero gas-insufflation duration (Di) that is sufficient to reach a fixed maximum-pressure threshold (Pₘₐₓ).

Depending on the embodiment concerned, the apparatus of the invention, namely the “IPPB apparatus”, may comprise one or more of the following features:
the regulated gas flowrate decreases in proportion to the increase in pressure until the maximum-pressure threshold (Pₘₐₓ) is reached.
the operating means are configured so that, during each inspiratory phase of the individual, they command the turbine to supply a regulated gas flowrate that decreases between an initial flowrate (Qᵢₙᵢₜ) and a final flowrate (Q_{fin}), where Qᵢₙᵢₜ>Q_{fin}.
the initial flowrate (Qᵢₙᵢₜ) is equal to: y·(Q_{reg}) where y is comprised between 0.5 and 2, preferably between 1 and 2.
the regulated gas flowrate (Q_{reg}) is chosen by the user, preferably via one or more setting keys for example or the like.
the setting key or keys are preferably one or more virtual keys displayed on a touchscreen, for example a screen forming part of a human-machine interface (HMI).
the final flowrate (Q_{fin}) is equal to a proportion (x %) of the initial flowrate (Qᵢₙᵢₜ) comprised between 0 and 70% of the initial flowrate (Qᵢₙᵢₜ), namely Q_{fin}=x %·Qᵢₙᵢₜ where x % is comprised between 0% (i.e. zero flowrate) and 70%.
the final flowrate (Q_{fin}) is equal to a proportion (x %) of the initial flowrate (Qᵢₙᵢₜ) comprised between 10 and 70% of the initial flowrate (Qᵢₙᵢₜ), namely Q_{fin}=x %·Qᵢₙᵢₜ where x % is comprised between 10 and 70%, and more preferably still, between 20 and 70%.
advantageously, the final flowrate (Q_{fin}) is equal to a proportion (x %) of the initial flowrate (Qᵢₙᵢₜ) comprised between 30 and 70% of the initial flowrate (Qᵢₙᵢₜ), namely Q_{fin}=x %·Qᵢₙᵢₜ where x % is comprised between 30 and 70%.
according to one embodiment, the final flowrate (Q_{fin}) is equal to a proportion (x %) of the initial flowrate (Qᵢₙᵢₜ) comprised between 40 and 60% of the initial flowrate (Qᵢₙᵢₜ).
according to one embodiment, the final flowrate (Q_{fin}) is equal to a proportion (x %) of the initial flowrate (Qᵢₙᵢₜ) comprised between 45 and 70% of the initial flowrate (Qᵢₙᵢₜ).
according to one embodiment, the final flowrate (Q_{fin}) is equal to a proportion (x %) of the initial flowrate (Qᵢₙᵢₜ) that is of the order of 50% of the initial flowrate (Qᵢₙᵢₜ).
as a preference, the target flowrate (Q_{target}) is equal to a proportion of the initial flowrate (Qᵢₙᵢₜ) and also to the increase in pressure in the patient circuit, according to

the following formula:

$${Q}_{target} = ( 100 \% - x \% ) \cdot {Q}_{init} \cdot ( {P}_{max} - {P}_{meas} ) / {P}_{max} + x \% \cdot {Q}_{init}$$

where:
   Q_{target} corresponds to the regulated flowrate value during the course of the inspiration concerned.
   Qᵢₙᵢₜ corresponds to the initial flowrate value, namely to a regulated flowrate (Q_{reg}), preferably the initial flowrate (Qᵢₙᵢₜ) is equal to: y·(Q_{reg}) where y is comprised between 0.5 and 2, preferably between 1 and 2.
   x % corresponds to the proportion of the initial flowrate (Qᵢₙᵢₜ) comprised between 0 and 70%, preferably between 30 and 70%, for example of the order of 50%,
   Pₘₐₓ is the fixed maximum pressure.
   Pₘₑₐₛ is the proximal pressure measured by the proximal pressure sensor.
the target flowrate corresponds to the theoretical flowrate value that the apparatus will use to regulate the flowrate delivered by the turbine.
the operating means are configured so that, during the course of the gas insufflation duration (Di) they command the turbine, i.e. its motor, so that the regulated gas flowrate supplied is a flowrate that decreases gradually, namely that decreases between the initial flowrate value (Qᵢₙᵢₜ) and the final flowrate value (Q_{fin}), where Qᵢₙᵢₜ>Q_{fin}.
during the course of the gas insufflation duration (Di), the turbine is operated using flowrate-control so as to supply the desired gradually-decreasing gas flowrate.
of course, the flowrate actually supplied by the turbine may exhibit slight variations or fluctuations around the desired flowrate value(s), for example fluctuations of the order of +/−5% or less.
the operating means are configured to command the electric motor of the turbine to supply the desired gas flowrate(s) and/or pressure(s).
the electric motor of the turbine is preferably a brushless and/or DC motor.
according to one particular embodiment, when a measured pressure (Pₘₑₐₛ) measured by the proximal pressure sensor is or becomes greater than or equal to the previously-fixed maximum-pressure threshold (Pₘₐₓ), (i.e. Pₘₑₐₛ=Pₘₐₓ), the operating means are configured to command the turbine to supply (or maintain) a plateau pressure (Pₚₗₐₜ) corresponding to the maximum-pressure threshold (Pₘₐₓ) so as to keep at least part of the external patient circuit at the plateau pressure (Pₚₗₐₜ), over the course of a given non-zero plateau duration (Dp).
duration-regulating means enable the user to set the plateau duration (Dp), preferably between 1 and 10 sec, typically between 0.5 and 5 sec.
during the course of the gas insufflation duration (Di), the turbine (i.e. its motor) is operated to control its flowrate, whereas thereafter, during the course of the plateau duration (Dp) it is operated to control pressure.
it comprises pressure-regulating means configured to allow the user to set the maximum-pressure threshold (Pₘₐₓ).
as a preference, the maximum-pressure threshold (Pₘₐₓ) is comprised between 10 and 50 cmH2O.
it comprises flowrate regulating means to enable the user to set the desired flowrate value (Q_{reg}), typically the initial flowrate value (Qᵢₙᵢₜ).
preferably, the flowrate value (Q_{reg}), typically the initial flowrate value (Qᵢₙᵢₜ), is comprised between 1 and 200 L/min, preferably between 3 and 150 L/min, and more preferably still, between 5 and 100 L/min.
the duration regulating means and/or the means for regulating pressure and/or for regulating flowrate comprise one or more buttons, keys or the like, particularly one or more buttons displayed on a touchscreen.
the operating means are configured to compare a measured proximal pressure value (Pₘₑₐₛ) measured by the proximal pressure sensor against the fixed maximum-pressure threshold (Pₘₐₓ) so as to determine the end of the gas insufflation duration (Di), namely the end of the insufflation of gas at decreasing flowrate.
an expiratory valve is arranged on the external patient circuit.
at the end of the gas insufflation duration (Di) or, if one is present, at the end of the plateau duration (Dp) following the gas insufflation period, the expiratory valve opens to discharge to the atmosphere at least some of the gas pressure present in the external patient circuit.
the expiratory valve opens in response to a drop in pressure in the internal gas circuit.
a pneumatic operating line for operating the expiratory valve is fluidically connected to the internal gas circuit between the turbine and the flowrate sensor.
the pneumatic operating line collaborates with the expiratory valve to control the opening or closing of said expiratory valve.
the opening of the expiratory valve enables gas, i.e. gas pressure, to be discharged from the external patient circuit to the ambient atmosphere and the pressure in all or part of the external patient circuit to decrease.
a pneumatic operating line is a pneumatic connection, typically a flexible tube, preferably made of polymer.
the operating means are connected to the proximal pressure sensor, namely the pressure sensor may, depending on the embodiment concerned, be electrically connected to said operating means and/or (directly) integrated into said operating means, so as to collaborate with said operating means.
as a preference, the proximal pressure sensor is integrated into the operating means, particularly borne by an electronic board that forms part of the operating means.
the proximal pressure sensor comprises a pressure measurement line that is connected to the patient circuit downstream of the expiratory valve.
the proximal pressure sensor is configured to enable measurement of the pressure within the external patient circuit between the expiratory valve and a breathing interface providing the gas exchanges with the patient, typically during the inspiratory phases and/or possibly during the expiratory phases of said patient.
the internal gas circuit comprises a passage, a duct or the like serving to carry the gas.
the breathing interface is a breathing mask, a tracheotomy tube or a mouthpiece.
the internal gas circuit comprises a gas outlet.
the external patient circuit is fluidically connected to said gas outlet of the internal gas circuit.
the external patient circuit comprises a flexible hose, typically made of polymer.
the external patient circuit may also comprise or employ additional fluidic-connection elements such as connectors, couplings or the like.
the external patient circuit comprises a single branch or is a single-branch circuit.
according to another embodiment, the external patient circuit may be a dual-branch circuit.
the operating means comprise at least one microprocessor.
said at least one microprocessor is arranged on at least one electronic board.
the operating means comprise at least an electronic board.
the motorized turbine comprises an electric motor.
the electric motor is electrically powered (110/220 V).
the operating means are configured to command the accelerations of the electric motor of the turbine and/or the decelerations or brakings of said motor. Deceleration means a braking or slowing of the motor, achieved for example by inverting the phases of the motor.
the nonreturn device comprises a check valve, a nonreturn valve or the like.
the nonreturn device is configured to oppose any backflow, namely any flow back towards the turbine, i.e. flowing in the opposite direction to the normal direction of circulation of the gas from the turbine towards the patient.
the expiratory valve is preferably a 3-way valve.
according to another embodiment, the expiratory valve may be a PEP valve.
the electric motor of the turbine is operated, i.e. commanded or controlled, by the operating means.
the pneumatic operating line comprises a flexible tube made of polymer and of small diameter, typically approximately 2 to 10 mm in diameter.
the peripheral casing or shell is formed from one or more sub-parts fixed together, for example by screw-fastening.
the peripheral casing or shell may be made of rigid polymer.
it comprises a graphical user interface or GUI, also known as a human-machine interface or HMI.
the GUI may comprise a display screen, preferably of the touchscreen type, and/or selection or validation keys, notably touch-sensitive keys displayed on the screen.
it comprises storage means, such as a RAM or EEPROM type memory, or a flash memory or the like.
the operating means command a slowing or a braking of the turbine when said operating means determine that the measured proximal pressure, typically measured by the proximal pressure sensor situated as close as possible to the patient, reaches a (pre-)set maximum-pressure value, namely the maximum-pressure threshold (Pₘₐₓ).
the operating means are configured to compare the measured proximal pressure value measured by the proximal pressure sensor against the (pre-)set maximum-pressure value, namely the maximum-pressure threshold (Pₘₐₓ), and from this to deduce the end of the gas insufflation duration (Di), the end of a maximum insufflation time (Tₘₐₓ), and/or the start of any potential plateau duration (Dp) there might be.
when a plateau duration (Dp) begins after the insufflation phase, this plateau duration is (previously-)fixed, i.e. set by the user (i.e. medical personnel), preferably via the GUI, and/or stored by the memory storage means.
the turbine motor is configured to be able to rotate at a rotational speed of as much as 50,000 rpm, or even 70,000 rpm.
the operating means comprise a timer enabling the durations to be determined or measured.
the maximum-pressure threshold (Pₘₐₓ) is (previously-)fixed, i.e. set by the user (i.e. medical personnel), preferably via the GUI or the HMI.
the maximum-pressure threshold (Pₘₐₓ) is stored in the memory-storage means.
the expiration valve, i.e. the expiratory valve, is configured to open automatically as soon as a depression is created in the internal circuit, i.e. upstream of the checkvalve that feeds into the valve, as a result of a deceleration (i.e. braking) of the turbine, i.e. namely when the turbine (motor) slows, particularly after the installation phase, or after any pressure plateau phase that might be present. This is possible thanks to the fluidic connection of the expiration valve operating line downstream of the turbine and upstream of the nonreturn device, namely between the turbine and the gas nonreturn device, and to the pneumatic operation (i.e. control by pressure) of said expiration valve by said operating line.
the expiration valve comprises a valve body comprising flexible sealing means.
the flexible sealing means comprise a membrane, a balloon or the like, preferably flexible and/or deformable, typically a membrane.
the pneumatic operating line controls the opening and/or the closing of the expiration valve by acting pneumatically, namely by supplying pressurized gas, on the flexible sealing means of the valve body.
the valve body of the expiration valve comprises at least one atmospheric-venting orifice.
the flexible sealing means are configured to allow or, conversely, to prevent, the circulation of the gaseous stream through the atmospheric-venting orifice, namely the discharging of gas to the atmosphere.
the flexible sealing means are arranged on a passage for a flow of gas passing through the valve body.
the flexible sealing means are configured to collaborate with at least part of the wall of the valve body so as to control the flow of gas or seal against the flow of gas, particularly in at least part of the wall of the gas flow passage arranged in the valve body.
the internal gas circuit comprises a gas outlet, for example borne by an outlet connector or endpiece or the like.
the external patient circuit is fluidically connected to the gas outlet of the internal gas circuit, for example is connected to the outlet connector or endpiece or the like of the internal gas circuit.
according to one embodiment, when a plateau duration follows the insufflation phase, the operating means control the turbine at the end of the insufflation phase so that during the course of the plateau duration Dp, the flowrate delivered by the turbine decreases gradually. A residual gas flowrate is maintained for the course of at least part of the plateau duration Dp, with the pressure moreover being maintained at Pₘₐₓ. During the course of the plateau duration Dp, the expiratory valve is closed so as to maintain at least part of the external patient circuit at the plateau pressure Pₚₗₐₜ, and at the end of the plateau duration Dp, the expiratory valve opens so as to discharge at least some of the gas pressure present in the external patient circuit.
the expiratory valve is kept closed during the course of the inspiration duration Di and during the course of the optional plateau duration Dp.

Moreover, the invention also relates to a method for providing respiratory therapy of the IPPB type to an individual who needs it, i.e. a patient, in which method use is made of a respiratory therapy apparatus (i.e. device or installation) as described hereinabove or hereinafter to supply respiratory gas to said individual, in which method, during each inspiratory phase of the individual, i.e. of the patient, there is supplied a regulated gas flowrate that decreases over the course of a non-zero gas-insufflation duration (Di) that is sufficient to reach a fixed maximum-pressure threshold (Pₘₐₓ).

The supply of the decreasing regulated gas flowrate is achieved by commanding the turbine of the apparatus, i.e. by the operating means.

In the context of the present invention:
a “pressure sensor” is the name given to any device or means able to determine a gas pressure.
a “flowrate sensor” is the name given to any device or means able to determine a gas flowrate, including indirectly via pressure measurements.
the terms “apparatus” and “device” are considered as being equivalent and interchangeable.
the terms “turbine”, “compressor”, “blower” or the like are considered as being equivalent and interchangeable.
the terms “valve” and “valve unit” are considered as being equivalent and interchangeable.
the terms “means” and “device” are considered as being equivalent and interchangeable, for example the terms “operating means” are considered as being

equivalent to and interchangeable with the terms “operating device”.
the terms “operated”, “commanded” and “controlled” are considered as being equivalent and interchangeable.
the terms “operate”, “command” and “control” are considered as being equivalent and interchangeable.
an individual's breathing is considered to comprise or to be formed of successive respiratory cycles, each respiratory cycle comprising an inspiratory phase followed by an expiratory phase.
during the course of an inspiratory phase, an individual inhales/draws into their lungs a respiratory gas, such as air or an air/O₂ mixture.
during the course of an expiratory phase, an individual exhales/expels from their lungs gas containing CO₂.

### BRIEF DESCRIPTION OF VIEWS OF DRAWINGS

The invention will now be better understood from the following detailed description, provided by way of non-limiting illustration, with reference to the appended figures, in which:

FIG. 1 is a process diagram of one embodiment of a respiratory therapy apparatus according to the invention.

FIG. 2 schematically depicts (in a partial exterior view) one embodiment of an apparatus according to the invention, such as that of FIG. 1, and its connection to the mains.

FIG. 3 schematically indicates curves of flowrate (Q) and pressure (P) obtained with an IPPB apparatus operated in accordance with the invention (INVENTION) and, by way of comparison, in accordance with the prior art (PRIOR ART).

FIG. 4 schematically indicates curves of flowrate (Q) and pressure (P) obtained with an IPPB apparatus operated in accordance with the invention, as illustrated in FIG. 3, but comprising a phase of maintaining a pressure plateau at the end of the insufflation duration.

### DETAILED DESCRIPTION

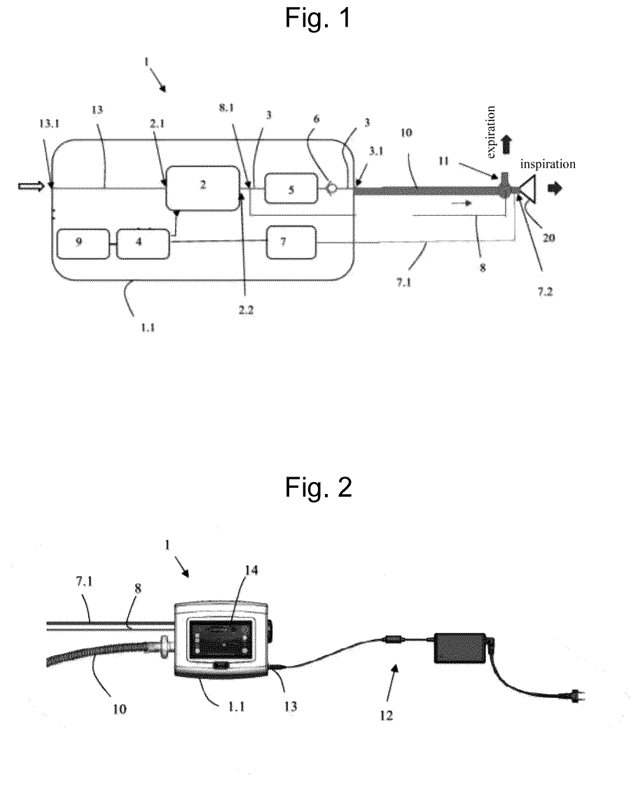

FIG. 1 and FIG. 2 schematically indicate one embodiment of a respiratory therapy apparatus or device 1 according to the invention, making it possible to provide intermittent positive pressure breathing assistance, hereinafter referred to as “IPPB therapy”, to an individual who needs it, namely typically to a patient who is to be treated, or the like.

As illustrated in the process diagram of FIG. 1, the respiratory therapy apparatus 1 or “IPPB apparatus” comprises a motorized turbine 2 (also referred to as (micro) blower, pump, compressor or the like) combined with a passive command system comprising an expiration valve 11, also referred to as an expiratory valve, and with proximal pressure measuring means, typically a proximal pressure sensor 7, as described hereinafter, with a view to delivering the IPPB therapy to the patient effectively and with improved patient comfort, notably in terms of limiting or reducing annoying noise, namely the noise generated during the functioning of the apparatus 1.

More specifically, the motorized turbine 2 comprises an electric motor, typically of the brushless type, which is operated, i.e. controlled, by operating means 4, typically by one (or more) microprocessor arranged on one (or more) electronic board. The turbine 2 is supplied with ambient air by an air inlet line 13, such as a duct, a passage or the like, which connects an air inlet 13.1 to the inlet 2.1 of the turbine 2.

The motorized turbine 2 has a conventional architecture, namely it schematically comprises an electric motor arranged in a protective housing, which is surmounted by a volute casing comprising an internal compartment in which a bladed wheel is arranged. The bladed wheel is borne by the shaft of the motor. It is rotationally driven by the shaft of the motor, when it is functioning, so as to draw in the respiratory gas, such as air, which then enters the internal compartment of the volute casing, then re-emerges therefrom before being sent to the patient.

The turbine 2 then supplies the respiratory gas, such as air, under pressure to an internal gas circuit 3 that serves to carry, i.e. convey, the respiratory gas delivered at the outlet 2.2 of the turbine 2 to a breathing interface 20, such as a breathing mask, a mouthpiece, or a tracheotomy tube, supplying the respiratory gas to the airways of the patient who is to be treated.

When the electric motor of the turbine 2 (i.e. its rotational speed) is controlled by the operating means 4, the motor shaft bearing the bladed wheel is rotationally driven, enabling the gas to be supplied at the desired flowrate, for example at the regulated flowrate (Q_{reg}), according to the control performed, as explained hereinafter.

Conversely, the operating means 4 can also control the rotational speed of the motor in order to brake or slow same. In that case, the rotational speed of the motor shaft bearing the bladed wheel decreases more or less rapidly and the supply of gas then decreases at a rate that correlates with the slowing/braking of the motor.

One example of a turbine or (micro) blower for a medical ventilation apparatus is given in EP2947328, but of course other types of turbine may be suitable.

Using a motorized turbine 2 to deliver the gas makes it possible to limit the noise generated while the apparatus 1 is functioning.

In order to convey the respiratory gas coming from the internal gas circuit 3, namely a circuit arranged inside the shell 1.1 of the apparatus 1, as far as the breathing interface 20, an external patient circuit 10 is provided, this typically being one (or more) flexible tube or duct that is fluidically connected to a gas outlet 3.1 of the internal gas circuit 3, typically a flexible tube made of polymer. The gas outlet 3.1 may be borne by an outlet coupling or connector arranged on the casing 1.1 of the apparatus 1. The external patient circuit 10 is fluidically coupled to said outlet coupling, for example by being push-fitted into it, screwed into it, or fixed in it using a bayonet system or any other suitable connection system, the flexible tube preferably comprising fluidic-connection means complementary to those of the outlet coupling.

In order to measure the gas flowrate within the apparatus 1, a flowrate sensor 5 is arranged on the internal gas circuit 3, namely downstream of the turbine outlet 2 and upstream of the gas outlet 3.1 of the gas circuit 3. The flowrate sensor 5 supplies the flowrate measurements to the operating means 4 where they are processed in order to regulate the operation of the turbine 2, typically the accelerations or braking of the electric motor thereof. The flowrate sensor 5 is electrically connected to the operating means 4, typically by electrical connections, such as electric cables or the like, so as to transmit to it the flowrate measurements taken.

These flowrate measurements can also be used to check the correct operation of the nonreturn device 6, e.g. checkvalve, by ensuring that there is no backflow flowing back towards the turbine 2 during the patient's expiratory phases, namely to check that the nonreturn valve 6 is providing fluidic sealing in the backflow direction.

The external patient circuit 10 for its part comprises the expiratory valve 11 which serves to control the discharges of gas exhaled by the patient and/or all or some of the gas pressure present in the patient circuit 10, particularly after the plateau phase as explained hereinafter, into the ambient atmosphere, given that this expiratory valve 11 allows the downstream part of the patient circuit 10 to be placed in communication with the outside, namely the ambient atmosphere, particularly at the end of the inspiratory phase and/or during the expiratory phases of the patient.

Moreover, in order to prevent backflow of gas/pressure and allow these to be discharged better via the expiratory valve 11, a nonreturn device 6, namely a checkvalve, a one-way valve or the like, is arranged on the internal gas circuit 3, downstream of the flowrate sensor 5 and upstream of the gas outlet 3.1 of the gas circuit 3. Such a nonreturn device 6 also offers the advantage of being simple to implement and of not requiring any particular operational control, and therefore also of being low in cost.

The presence of this nonreturn device 6, typically a checkvalve, thus makes it possible to improve the functioning of the apparatus 1, typically the opening of the expiratory valve 11 during the expiratory phases, as explained hereinbelow.

The expiratory valve 11, particularly the opening and/or closing thereof, is/are commanded or controlled by a pneumatic operating line 8, such as a flexible duct, which is fluidically connected 8.1 to the internal gas circuit 3 between the turbine 2 and the flowrate sensor 5. The pneumatic operating line 8 collaborates with the expiratory valve 11 by supplying it with a gas pressure that serves to control the opening or the closing of said expiratory valve 11, namely the extent to which it is open/closed.

As a preference, the opening and/or the closing of said expiratory valve 11 is/are controlled by flexible sealing means on which the gas pressure supplied by the pneumatic operating line 8 acts, as explained hereinbelow.

Finally, the external patient circuit 10 also comprises a proximal pressure sensor 7 arranged in such a way as to enable measurement of the pressure, i.e. the instantaneous pressure, within the patient circuit 10 and close to 7.2 the expiratory valve 11, typically between the expiratory valve 11 and the breathing interface 20.

This proximal pressure sensor 7 makes it possible to detect the patient's demands for gas, namely their inhalations, but also whether the fixed maximum pressure threshold (Pₘₐₓ) has been reached at the end of the inspiratory phrase, as detailed hereinbelow.

The proximal pressure sensor 7 is connected to the operating means 4, namely collaborates therewith, so that the latter can operate the turbine 2 on the basis of all or some of the measurements taken by the proximal pressure sensor 7. As a preference, the proximal pressure sensor 7 is directly integrated into the operating means 4, for example borne by an electronic board of said operating means 4.

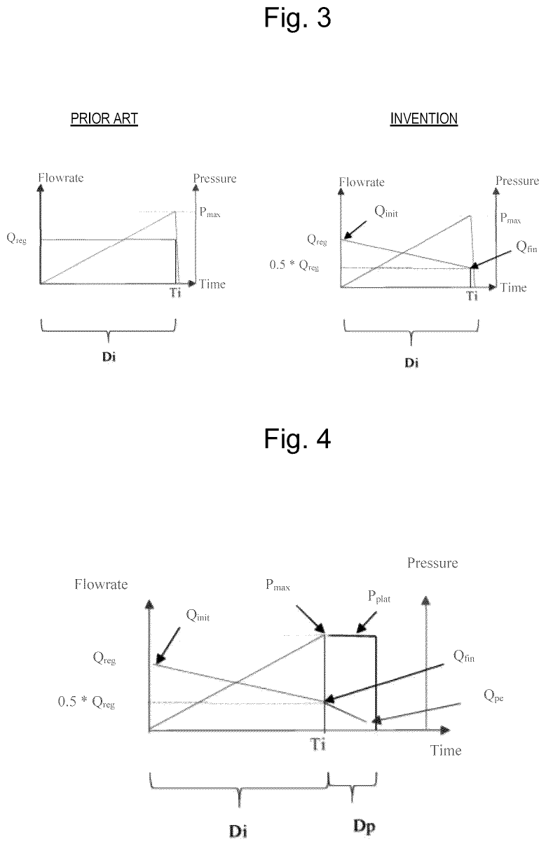

The use of an operationally controlled motorized turbine 2 ensures improved comfort both in terms of the lower noise generated by the apparatus 1 during an IPPB treatment and in terms of the adaptability of the profile of the flow delivered to the patient, for example a flowrate of “rectangular” shape as illustrated in FIG. 3, or another.

Moreover, the measurement of the proximal pressure, using the proximal pressure sensor 7 provides the device 1 with additional performance thanks to the high precision of the pressure measurements taken, and also comfortable use for the patient, thanks to better detection of the patient's respiratory effort, making it possible to obtain effective, or even (near-)optimal synchronization between the patient's demand for gas and the supply of gas by the turbine during the inspiratory phases and, conversely, the discharging of the exhaled gases via the expiratory valve 11.

As illustrated in FIG. 2, the apparatus 1 also comprises regulating or selection means, such as virtual keys displayed by the display, i.e. digital screen 14, of a graphical user interface, or GUI, 9. The display screen 14 is preferably of the touchscreen and/or colour display screen type. It comprises selection or validation keys, notably touch-sensitive keys displayed on the screen.

The turbine 2, the operating means 4, the internal circuit 3, the flowrate sensor 5 and the nonreturn device 6 are arranged in the peripheral casing or shell 1.1 of the apparatus 1, for example a casing made of a rigid polymer or some other suitable material.

In general, electrical power supply means, such as a mains (110/220 V) electrical connection are also provided, these enabling electrical power to be supplied to the apparatus 1, particularly to all the components that require electrical power in order to function, such as the operating means 4, the GUI 9, the screen 14, the sensors 5, 7, etc. As a preference, as illustrated in FIG. 2, the electrical power supply means may comprise a unit 12 comprising one (or more) electrical cables with a current transformer and a plug for connecting to the (110/220 V) mains and that can be connected to an electrical connector 13 of the apparatus 1.

Furthermore, the apparatus 1 may also comprise means for starting and/or stopping it, such as an “On/Off” key or the equivalent.

While the apparatus 1 is functioning, during the course of the patient inspiratory or insufflatory phases, the turbine 2 is operated in such a way as to deliver a regulated flowrate that decreases according to the control parameter(s) or setting(s) input, fixed or selected by the user via the GUI 9, particularly a maximum-pressure value, also referred to as maximum-pressure threshold (Pₘₐₓ), as illustrated in FIG. 3 and FIG. 4 and possibly a maximum inspiration time (Tₘₐₓ), namely a maximum duration for the course of which gas is delivered to the patient during each inspiratory phase of duration Di. According to other embodiments, other parameters, such as an inspiratory and/or expiratory gradient, a trigger sensitivity, a therapy time, etc. may also be selected.

More specifically, the operating means 4, typically one (or more) microprocessor, are configured to command the turbine 2, typically the electric motor of the turbine, during each inspiratory phase of the patient, to first of all supply a controlled/desired gas flowrate (Q_{reg}), typically an air flowrate, over the course of a non-zero gas insufflation duration (Di) that is sufficient to achieve a previously-fixed maximum-pressure threshold (Pₘₐₓ), for example comprised between 10 and 50 cmH2O.

Thus, FIG. 3 provides a comparison of curves of flowrate (Q)/pressure (P), as a function of time, obtained with the IPPB apparatus 1 controlled for flowrate in accordance with the prior art (“PRIOR ART”) or in accordance with the invention (“INVENTION”).

In both instances, during inspiration in each respiratory cycle of the patient, the turbine 2 of the apparatus 1 delivers the desired gas flowrate Q_{reg}, which according to the Prior Art (left-hand graph) is constant, or which according to the invention (right-hand graph) decelerates/decreases as a function of the regulation performed via the interface or GUI.

The regulated flowrate is delivered to the patient through the flowrate sensor 5 and the checkvalve 6 of the apparatus of FIG. 1.

The flowrate is regulated using closed-loop control by the operating means 4 such as a computer, on the basis of flowrate measurements taken at the outlet/downstream of the turbine 2. The speed of the turbine 2 can thus be adapted by the operating means 4 to suit the desired flowrate profile. The regulating control, for example of the proportional integral derivative (PID) type, makes it possible to maintain a minimal difference between the desired flowrate profile and the flowrate profile resulting from the adaptation of the turbine speed and to do so whatever the resultant pressure provided that this pressure remains below the maximum-pressure threshold Pₘₐₓ at which inhalation is cut off.

When the flowrate is delivered to the patient, the pressure P in the external patient circuit 10 and therefore in the patient's lungs increases gradually, for example as here linearly, although a non-linear increase is also possible, until the previously-fixed and memory-stored maximum pressure Pₘₐₓ is reached.

Throughout the duration of the patient inspiratory phase, namely throughout the duration Di, the expiratory valve 11 is closed.

When the measured instantaneous pressure (Pₘₑₐₛ) measured by the proximal pressure sensor 7 reaches the previously-fixed maximum-pressure threshold (Pₘₐₓ), i.e. Pₘₑₐₛ=Pₘₐₓ, the operating means 4 stop operating the turbine 2 to control flowrate.

At that moment, the turbine 2 is no longer operated to control flowrate and slows, leading to a drop in the flowrate of gas supplied and also, in the conditions of FIG. 3, to a rapid drop in pressure in the external patient circuit 10 and therefore in the patient's lungs because at the end of the insufflation phase Di and/or of the maximum insufflation time Ti, the expiration valve 11 opens, causing gas to be discharged to the atmosphere.

In both instances, the gas-insufflation duration Di corresponds to the time needed for the gas pressure in the external patient circuit 10, and therefore also in the patient's lungs, to increase gradually until a desired maximum pressure, namely the previously-fixed maximum-pressure threshold Pₘₐₓ is reached. Here, the increase in gas pressure is linear, although as already stated, it could be non-linear given that the increase in pressure is the result of the flowrate of gas supplied to the patient's lungs, namely the volume of gas that enters therein, which may be (highly) variable notably according to the compliance and/or the resistance of the patient's lungs.

In the left-hand graph (Prior Art), which illustrates control of flowrate in accordance with the prior art, the regulated flowrate Q_{reg} set by the user and delivered to the patient by the turbine 2 is kept constant during the course of the gas insufflation duration Di, this being for example a regulated flowrate Q_{reg} comprised between 5 and 100 L/min, which is manifested by a curve of “rectangular” shape.

By contrast, in the right-hand graph (Invention), which illustrates control of flowrate in accordance with the invention, the regulated flowrate Q_{reg} set by the user and delivered to the patient by the turbine 2 is not kept constant during the course of the gas insufflation duration Di, but is controlled in such a manner as to decrease, namely to decrease gradually between an initial flowrate initial value Qᵢₙᵢₜ and a final flowrate final value Q_{fin}.

Once again, the regulated flowrate Q_{reg} corresponding to the initial flowrate Qᵢₙᵢₜ may be comprised between 5 and 100 L/min.

The regulated gas flowrate therefore decreases in proportion to the increase in pressure until the maximum-pressure threshold (Pₘₐₓ) is reached.

Here, the decrease in flowrate is linear but it could have some other shape, for example a curved shape, such as a second-degree or higher-degree polynomial, or some other shape.

In order to effect decreasing-flowrate control in accordance with the invention (i.e. the decreasing gradient in the right-hand graph), the initial flowrate (Qᵢₙᵢₜ) corresponds to the regulated flowrate at the start of inspiration, for example 40 L/min, and will be reduced gradually until the flowrate reaches the desired final flowrate (Q_{fin}), which is lower than the initial flowrate (namely: Q_{fin}<Qᵢₙᵢₜ).

As a preference, the final flowrate (Q_{fin}) corresponds or is equal to a proportion (x %) of the initial flowrate (Qᵢₙᵢₜ), namely Q_{fin}=x %·Qᵢₙᵢₜ, for example a proportion (x %) comprised between 0 and 70% of the initial flowrate (Qᵢₙᵢₜ), advantageously between 30 and 70% of the initial flowrate (Qᵢₙᵢₜ). By way of example, the final flowrate (Q_{fin}) is set here to approximately 50% (x %=50%) of the regulated flowrate at the start of the insufflation phase, namely to 50% of the initial flowrate (Qᵢₙᵢₜ).

According to one embodiment according to the invention, the reduction in flowrate is also preferably proportional to the increase in pressure in the circuit and in the lungs, considering the proportion with respect to the maximum pressure in accordance with the following formula:

$${Q}_{target} = ( 100 \% - x \% ) \cdot {Q}_{init} \cdot ( {P}_{max} - {P}_{meas} ) / {P}_{max} + x \% \cdot {Q}_{init}$$

where:
Q_{target} corresponds to the regulated flowrate value during the course of the inspiration concerned.
Qᵢₙᵢₜ corresponds to the initial flowrate value, namely to a regulated flowrate (Q_{reg}), preferably the initial flowrate (Qᵢₙᵢₜ) is equal to: y·(Q_{reg}) where y is comprised between 0.5 and 2, preferably between 1 and 2.
x % corresponds to a proportion (for example x %=50%) with respect to the initial flowrate.
Pₘₐₓ is the fixed maximum pressure
Pₘₑₐₛ is the proximal pressure measured by the pressure sensor

In this case:
If Pₘₑₐₛ=0 then Q_{target}=Qᵢₙᵢₜ at the start of inspiration, namely to the initial flowrate (Qinit)
If Pₘₑₐₛ=Pₘₐₓ then Q_{target}=x %·Qᵢₙᵢₜ at the end of inspiration.

According to the invention (the right-hand graph in FIG. 3), the act of controlling the turbine 2 so that it delivers a flowrate that decreases/decelerates during the insufflation phase (decreasing gradient) makes it possible to improve patient comfort because this profile is closer to patient physiology and, moreover, makes it possible to deliver a greater volume for the same maximum pressure. Having a lower flowrate at the end of inspiration implies that the pressure resulting from the resistance of the patient and any interface accessories there might be is lower. Therefore, for the same attained pressure, a lower proportion is the result of the resistance of the patient which means that the lungs can be filled better. This may also cause the patient to adhere to their treatment better.

Ultimately, this leads to improvements to the overall treatment in comparison with control according to the Prior Art (left-hand graph in FIG. 3), namely control with a “rectangular” curve whereby a constant flowrate is delivered.

Moreover, FIG. 4 schematically indicates curves of flowrate (Q) and pressure (P) obtained with an IPPB apparatus operated in accordance with the invention, as illustrated in FIG. 3, but further comprising a phase of maintaining a pressure plateau (Pₚₗₐₜ) at the end of the insufflation duration Di.

The gas insufflation phase (Di) is identical to that illustrated in FIG. 3 and will not be explained in detail because details thereof can be appreciated simply by referring to the explanations hereinabove.

In this embodiment, the major difference lies at the end of the insufflation phase, i.e. after the insufflation duration Di. Specifically, as visible in FIG. 4, after the insufflation duration Di at decreasing flowrate, i.e. at the end of the maximum insufflation time (Tₘₐₓ), in this embodiment, the pressure is maintained (approximately) constant over the course of a non-zero plateau duration (Dp) so as to keep at least part of the external patient circuit 10 at a plateau pressure (Pₚₗₐₜ) which is equal to the previously-fixed maximum-pressure threshold (Pₘₐₓ), i.e. Pₚₗₐₜ=Pₘₐₓ.

For example, the plateau duration (Dp) may be comprised between 0.1 and 10 sec, typically between 0.5 and 5 sec.

For this, the operating means 4 command the turbine 2 to supply a plateau pressure (Pₚₗₐₜ) corresponding to the maximum-pressure threshold (Pₘₐₓ) once this threshold has been reached.

In other words, according to the invention, during each inspiratory phase of the patient, the operating means 4 first of all command the turbine 2 to control flowrate over the course of the gas-insufflation duration (Di), and then, when the plateau pressure (Pₘₐₓ) has been reached, namely at the end of the gas-insufflation duration Di, they command it to control pressure over the course of the plateau duration (Dp).

Throughout the duration of the patient inspiratory phase, namely throughout the durations Di and Dp, the expiratory valve 11 remains closed.

Thus, once the maximum-pressure threshold Pₘₐₓ has been reached, the gas pressure in the patient circuit 10 can be maintained at the maximum-pressure threshold value Pₘₐₓ so as to obtain a pressure plateau (Pₚₗₐₜ) for the pressure prevailing in the patient circuit 10, particularly in its downstream part close to the patient and their airways.

This plateau pressure Pₚₗₐₜ is kept (approximately) constant over the course of the plateau duration Dp, whereas, in the same time interval, the flowrate continues to decrease gradually from the final value Q_{fin} reflecting the flowrate at the end of the gas-insufflation duration Di, to a lower flowrate value, namely a plateau-end flowrate value Qₚₑ, for example a Qₚₑ value that is zero or close to zero (i.e. 0 L/min). This gradual, over the course of the plateau duration Dp, reduction in the flowrate corresponds to the slowing or braking of the turbine 2 which is now operated to control pressure (rather than flowrate) in order to maintain the plateau pressure Pₚₗₐₜ.

Thanks to the application of this pressure plateau (Plateau), patient comfort and the effectiveness of the patient's overall treatment can be (even further) improved, given that the pressure-plateau phase makes it possible to increase the volume of gas, i.e. air, delivered to the patient and thus to improve the propagation of the gas into the patient's lungs, all the while without increasing the maximum cut-off pressure, namely the maximum pressure (i.e. Pₘₐₓ) delivered, and therefore while limiting any barotrauma-type side effects of the treatment.

Thanks to the pressure-plateau phase, a member of the medical-care team, i.e. a clinician or similar, can adapt the patient's therapy to obtain the best possible trade-off between maximum pressure and treatment effectiveness.

According to the invention, the operating means 4 of the apparatus 1 therefore make a comparison between the measurements of proximal pressure Pₘₑₐₛ (i.e. instantaneous pressure) coming from the proximal pressure sensor 7 and the previously-fixed maximum-pressure threshold Pₘₐₓ, and potentially (depending on the desired embodiment) trigger a plateau phase whereby the plateau pressure Pₚₗₐₜ is maintained.

Moreover, given that at the end of the plateau duration Dp the turbine 2 ceases to be operated for the control of pressure, there is a sharp drop in the pressure prevailing in the internal circuit 3 of the apparatus 1, particularly at the tapping 8.1 for the operating line that operates the expiratory valve 11, and therefore within the operating line that operates the expiratory valve 11 itself. This sharp drop in pressure will then cause the expiratory valve 11, which was hitherto closed, to open so as to discharge at least some of the gas pressure present in the patient circuit 10 to the external atmosphere and thus obtain a drop in pressure in the patient circuit 10 and in the lungs of the patient, so as to allow them to exhale the CO₂-rich exhaled gas.

The residual gas flowrate maintained over the course of at least part of the plateau duration Dp can decrease gradually following a linear or non-linear profile, for example a substantially parabolic or other profile.

In general, the control parameter(s) for the apparatus 1, particularly the maximum-pressure threshold (Pₘₐₓ), the plateau duration (Dp) for any plateau there might be and/or the regulated flowrate(s) Q_{reg}, particularly Qᵢₙᵢₜ, may be stored in memory-storage means of the apparatus 1, such as a flash memory, a RAM, an EEPROM or the like. These parameters can be regulated/set via pressure-, flowrate- and/or duration-regulating means, such as one or more regulating buttons or keys, for example virtual or analogue keys displayed by the GUI.

In all cases, as illustrated in FIG. 1, the gas, such as air, coming from the turbine 2 while it is functioning, is delivered to the patient after having been conveyed by the internal gas circuit 3, and therefore via the flowrate sensor 5 and the nonreturn device 6 (i.e. checkvalve or the like).

The pneumatic operating line 8, i.e. flexible tube or the like, that serves to operate the expiratory valve 11 is connected to the internal gas circuit 3 downstream (at 8.1) of the outlet of the turbine 2, which means that the expiratory valve 11 can be closed typically by action on the flexible sealing means of the expiratory valve 11 while the patient is inhaling, namely over the course of the inspiration and/or plateau phases (i.e. over the course of the durations Di and/or Dp) according to the embodiment concerned, thanks to the gas pressure coming from the turbine 2 that travels via the pneumatic operating line 8 as far as the expiratory valve 11.

As a preference, the flexible sealing means of the expiratory valve 11 comprise a balloon or a flexible/deformable membrane or the like, and an atmospheric-venting orifice or vent in communication with the atmosphere so as to control the flowrate of gas, namely to block any gas from leaving via said atmospheric-venting orifice thereby preventing any gas from leaving during the inspiratory phases, or conversely to uncover this orifice to a greater or lesser extent so as then to allow gas to pass to the atmosphere and therefore to leave the external patient circuit 10. Such an architecture is conventional.

In other words, the flexible sealing means are arranged in the valve body and collaborate with part of the wall of said valve body in the manner of a valve shutter and valve-shutter seat so as to control the flow of gas through the valve body and the release of gas to the atmosphere.

In one embodiment, the expiratory valve 11, i.e. expiration valve, may further comprise a fluidtight zone comprised between the membrane and the upper body of the valve unit 11 which allows operation as a function of the pressure sent to the fluidtight zone via the operational-control tapping.

Moreover, the proximal pressure measurement(s) taken by the proximal pressure sensor 7 reflects (reflect) the pressure prevailing in the patient circuit 10 as close as possible to the patient (at 7.2), namely in the immediate vicinity of the patient's airways.

When the proximal pressure measured as close as possible to the patient reaches the (pre-)set maximum pressure value, namely the previously-fixed maximum-pressure threshold (Pₘₐₓ), or else when a (previously-)fixed or set maximum inspiration time (Tₘₐₓ) has been reached, the operating means 4 control the turbine 2, more specifically the electric motor thereof, so that this turbine slows and/or brakes, namely so that the rotations of the motor 2 decrease and/or stop, but in such a way as possibly to keep a positive pressure in the circuit 10 at the plateau-pressure value Pₚₗₐₜ when such a plateau is desired.

As has already been stated, the comparisons between the measured proximal pressure and previously-fixed maximum-pressure, namely the previously-fixed maximum-pressure threshold (Pₘₐₓ) values are made by the operating means 4, typically by a microprocessor.

Moreover, the operating means 4 may further comprise a timer or the like for determining the durations or other time periods.

Because of the presence of the nonreturn device 6, any reduction or drop in pressure is not transmitted directly to the patient circuit 10 but remains in the internal circuit 3 and in the operating line 8 that operates the expiration valve 11, thereby allowing the valve 11 to open, after the insufflation or plateau phase, since the pressure prevailing in the balloon or on the membrane of the valve 11 decreases, thus allowing gas to escape to the atmosphere, and therefore allowing the patient to exhale through said expiratory valve 11 which then is in fluidic communication with the atmosphere, via its atmospheric-venting orifice, given that the balloon or the membrane is no longer sealing against the passage of gas.

Patient expiration therefore progresses passively through the expiration valve 11 of the patient circuit 10 and to the atmosphere, i.e. through the valve body and the atmospheric-venting orifice.

More generally, in the apparatus of the invention, the use of the proximal pressure for synchronizing the patient's inspiratory and expiratory phases allows the system better selectivity which is no longer influenced by the flowrate coming from the turbine 2 and by the pneumatic resistances of the patient circuit 10.

The apparatus 1 detects the patient's respiratory effort more precisely and with greater sensitivity, making it possible to improve the IPPB treatment notably as a result of better synchronization between the delivery of gas and the patient's respiratory effort.

In the case of the use of PID control regulation, the switch from regulating flowrate to regulating pressure requires the resetting of certain parameters, such as the integral factor, to prevent the turbine speed from dropping during the phase of loading the integral factor.

In general, the respiratory therapy apparatus according to the invention is well suited to the supply of respiratory therapy of the IPPB type to patients who need it, typically to individuals suffering from respiratory problems or conditions that need their bronchial drainage, their respiratory function and their lung recruitment, particularly recruitment of those regions of the lungs that are poorly ventilated or unventilated, to be improved.

What is claimed is:

1. Respiratory therapy apparatus (1) for supplying intermittent positive pressure breathing (IPPB) assistance to an individual, comprising:
a peripheral casing or shell (1.1),
a motorized turbine (2) for supplying a respiratory gas under pressure,
an internal gas circuit (3) for conveying the respiratory gas delivered by the turbine (2),
a flowrate sensor (5) arranged on the internal gas circuit (3),
a nonreturn device (6) arranged in the internal gas circuit (3) downstream of the flowrate sensor (5),
an external patient circuit (10) that can be fluidically connected to the internal gas circuit (3) to convey the respiratory gas coming from the internal gas circuit (3),
a proximal pressure sensor (7) arranged to allow the measuring of the pressure within the external patient circuit (10), and
operating means (4) connected to the proximal pressure sensor (7) and to the
motorized turbine (2) and configured to command the turbine (2) as a function of at least one pressure measurement taken by the proximal pressure sensor (7) and of at least one flowrate measurement taken by the flowrate sensor (5),
and wherein the turbine (2), the operating means (4), the internal circuit (3), the flowrate sensor (5) and the nonreturn device (6) are arranged in the peripheral casing or shell (1.1),
characterized in that the operating means (4) are configured so that, during each inspiratory phase of the individual, they command the turbine to supply a regulated gas flowrate that decreases, over the course of a non-zero gas-insufflation duration (Di) that is sufficient to reach a fixed maximum-pressure threshold (Pₘₐₓ).

2. Apparatus according to claim 1, characterized in that it further comprises an expiratory valve (11) arranged on the external patient circuit (10), and a pneumatic operating line (8) for operating the expiratory valve (11) and that is fluidically connected (8.1) to the internal gas circuit (3) between the turbine (2) and the flowrate sensor (5) and collaborates with the expiratory valve (11) to control the opening or closing of said expiratory valve (11).

3. Apparatus according to claim 2, characterized in that the proximal pressure sensor (7) is arranged to enable measurement of the pressure within the external patient circuit (10) via a pressure-measurement line (7.1) that is connected to the patient circuit (10) downstream (7.2) of the expiratory valve (11).

4. Apparatus according to claim 1, characterized in that when a measured pressure (Pₘₑₐₛ) measured by the proximal pressure sensor is greater than or equal to the previously-fixed maximum-pressure threshold (Pₘₐₓ), the operating means (4) are configured to command the turbine to supply a plateau pressure (Pₚₗₐₜ) corresponding to the maximum-pressure threshold (Pₘₐₓ) so as to keep at least part of the external patient circuit at said plateau pressure (Pₚₗₐₜ), over the course of a given non-zero plateau duration (Dp).

5. Apparatus according to claim 1, characterized in that the regulated gas flowrate decreases in proportion to the increase in pressure until the maximum-pressure threshold (Pₘₐₓ) is reached.

6. Apparatus according to claim 1, characterized in that the operating means (4) command a slowing or a braking of the turbine (2) when said operating means (4) determine that the measured proximal pressure has reached the maximum-pressure threshold (Pₘₐₓ).

7. Apparatus according to claim 1, characterized in that the operating means (4) are configured so that, during each inspiratory phase of the individual, they command the turbine to supply a regulated gas flowrate that decreases between an initial flowrate (Qᵢₙᵢₜ) and a final flowrate (Q_{fin}), where Qᵢₙᵢₜ>Q_{fin}.

8. Apparatus according to claim 7, characterized in that the final flowrate (Q_{fin}) is equal to a proportion (x %) of the initial flowrate (Qᵢₙᵢₜ) comprised between 0 and 70% of the initial flowrate (Qᵢₙᵢₜ).

9. Apparatus according to claim 8, characterized in that the target flowrate (Q_{target}) is equal to a proportion of the initial flowrate (Qᵢₙᵢₜ) and also to the increase in pressure in the patient circuit (10), according to the following formula:
$${Q}_{target} = ( 100 \% - x \% ) \cdot {Q}_{init} \cdot ( {P}_{max} - {P}_{meas} ) / {P}_{max} + x \% \cdot {Q}_{init}$$
where:
Q_{target} corresponds to the regulated flowrate value during the course of the inspiration concerned.
Qᵢₙᵢₜ corresponds to the initial flowrate value, namely to a regulated flowrate (Q_{reg}), preferably the initial flowrate (Qᵢₙᵢₜ) is equal to: y·(Q_{reg}) where y is comprised between 0.5 and 2, preferably between 1 and 2.
x % corresponds to the proportion of the initial flowrate (Qᵢₙᵢₜ) comprised between 0 and 70%, preferably between 30 and 70%, for example of the order of 50%,
Pₘₐₓ is the fixed maximum pressure.
Pₘₑₐₛ is the proximal pressure measured by the proximal pressure sensor.

10. Apparatus according to claim 1, characterized in that the maximum-pressure threshold (Pₘₐₓ) is comprised between 10 and 50 cmH2O and/or the initial flowrate (Qᵢₙᵢₜ) is comprised between 1 and 200 L/min.

11. Apparatus according to claim 8, characterized in that the proportion (x %) is comprised between 30 and 70% of the initial flowrate (Qᵢₙᵢₜ).

12. Apparatus according to claim 10, characterized in that the initial flowrate (Qᵢₙᵢₜ) is comprised between 3 and 150 L/min.

13. Apparatus according to claim 1, characterized in that the operating means (4) comprise at least one microprocessor.

14. Apparatus according to claim 1, characterized in that the motorized turbine (2) comprises an electric motor operated by the operating means (4).

15. Apparatus according to claim 1, characterized in that the proximal pressure sensor (7) is configured to enable measurement of the pressure within the external patient circuit (10) between the expiratory valve (11) and a breathing interface (20) providing the gas exchanges with the patient.
